(19) United States
(12) Patent Application Publication
(10) Pub. No.: US 2011/0230867 A1
(43) Pub. Date: Sep. 22, 2011

(54) LAPAROSCOPIC NEEDLE HOLDER

(75) Inventors: Simon HIRSCHFELD, Hamburg (DE); Markus BAUER, Hamburg (DE)

(73) Assignee: OLYMPUS WINTER & IBE GMBH, Hamburg (DE)

(21) Appl. No.: 13/044,980

(22) Filed: Mar. 10, 2011

(30) Foreign Application Priority Data
Mar. 18, 2010 (DE) 102010011926.1-35

(51) Int. Cl. A61B 17/00

(52) U.S. Cl. 606/1

(57) ABSTRACT

A laparoscopic needle holder includes a shaft embodied as a tube, at the distal end of which clamping jaws are arranged. The clamping jaws can be actuated by a rod running through the shaft, the rod is longitudinally displaceable by a handle arranged on the proximal end of the shaft. The laparoscopic needle holder is characterized in that the shaft has a straight proximal shaft section and a curved distal shaft section, and the two sections are detachably attached to one another.

### BACKGROUND

The invention relates to a laparoscopic needle holder, a method for the production thereof and a curved distal shaft section of the needle holder.

Laparoscopic needle holders in the standard design typical of today are shown in U.S. Pat. No. 5,413,583 A and U.S. Pat. No. 5,951,587 A. They have clamping jaws on the distal end of an elongated thin shaft tube, with which surgical needles can be held and moved for the purpose of suturing. In the known instruments, the handle is typically embodied as an inline handle, that is, with an extension essentially in the direction of the shaft tube. This handle form is well suited for exerting the necessary high holding forces as well as for the typical suturing movement. With the elongated shaft, generic instruments are suitable for insertion through a laparoscopic port into the abdominal cavity in order to be able to suture there under endoscopic observation.

Recently, a laparoscopic technique has become accepted as is shown in DE 202009007592 U1. Work is thereby carried out with two instruments through a port. For the reasons explained in this document, it is advantageous thereby if the shafts of the instruments are embodied in a curved manner in their distal section, since then the instruments work tip against tip, but can nevertheless bear against one another in a parallel manner in the region of the common port.

### SUMMARY

An object of the present invention is to embody a laparoscopic needle holder in an inexpensive manner for use in a commonly used port.

This object is attained by providing a laparoscopic needle holder including a tubular shaft having a proximal shaft section and a distal shaft section. The proximal shaft section and the distal shaft section are detachably attached. Clamping jaws are disposed at the distal end of the shaft, and a handle is disposed at the proximal end of the shaft. A rod having a cam that couples with the handle is inserted into the distal shaft section, and the distal shaft section is bent to form the curved distal shaft section, before the proximal shaft section is attached to the distal shaft section.

According to one aspect of the invention, the needle holder is embodied with a shaft that has a curved distal shaft section and a straight proximal shaft section, and thus, it is excellently suitable for the purposes cited in the latter specification. The two shaft parts are attached to one another in a detachable manner and therefore can be produced separately. This makes production much easier and cheaper, since the straight proximal shaft section, together with the handle, can be taken from a generic needle holder. Only the curved distal shaft section has to be produced separately, which reduces the problems due to the short length of this shaft section.

The two shaft sections can be connected to one another in any desired manner, but advantageously via a coupling that is disposed at a distal end of the proximal shaft section and that can accommodate the clamping jaws. Therefore, alternatively, a clamping jaw can be arranged directly on the distal end of the straight proximal shaft section. The unit of the straight proximal shaft section and the handle can therefore be used together with clamping jaws as a straight needle holder according to the prior art, as well as with the connected curved distal shaft section as a needle holder for use with other instruments in a common port.

The clamping jaws can be attached to the distal end of the distal shaft section in different ways, for example, removable for cleaning purposes or rotatable. However, if the clamping jaws are permanently attached, e.g., welded, to the distal section, this is advantageous because the construction and the production are greatly simplified.

A thickened cam, e.g., in the usual form of a sphere, is provided at the proximal end of the rod in a manner known per se, which cam is used for coupling with the moving parts of the handle. This cam can be attached to the end of the rod in various ways, e.g., by means of welding. However, the cam can be advantageously attached to the rod such that a space between the proximal end of the rod and the cam is adjustable at an individually adjusted spacing, which means that differences in length of the rod can be compensated for, which otherwise would impede the exact adjustment of the ranges of movement of the clamping jaws and the handle.

Advantageously, the cam can be screwed onto and welded to the rod. This firstly renders possible a length adjustment with the threaded joint in order subsequently to be able to fix the threaded joint by means of welding.

An advantageous method for producing the needle holder according to the invention is summarized below. The laparoscopic needle holder includes a shaft having a proximal shaft section and a distal shaft section that are detachably attached, clamping jaws, and a rod having a cam for coupling with a handle. According to the method, a prefabricated mounting unit including clamping jaws and the rod are inserted into the distal shaft section, and the clamping jaws are attached to the distal end of the distal shaft section. The distal shaft section is bent with the rod inserted to form the curved distal shaft section. Then, the proximal shaft section is attached to the distal shaft section. Accordingly, firstly the clamping jaws and rod are connected. Then the rod is inserted into the shaft and the clamping jaws are attached to the distal end of the distal shaft part. This is thereby still straight and is only curved afterwards, together with the rod on the inside. Subsequently, the distal shaft section is attached to the proximal shaft section. This sequence of steps has proven to be particularly useful, since it ensures a high production quality at low cost.

Advantageously, the cam, which is usually embodied as a sphere, is adjusted at the desired spacing on the rod by screwing and is attached by means of subsequent welding. This sequence of steps has also proven to be particularly advantageous and cost-effective.

In addition, an assembly including the curved distal shaft section of a laparoscopic needle holder can also be provided.

### BRIEF DESCRIPTION OF THE DRAWINGS

The invention is shown diagrammatically and by way of example in the drawing.

FIG. 1 is a side view of a needle holder according to an embodiment of the invention;

FIG. 2 is a view of only the assembly containing the distal shaft section of the needle holder of FIG. 1; and

FIG. 3 is a side view of an assembly that can be used instead of the construction of FIG. 2.

### DETAILED DESCRIPTION OF EMBODIMENTS

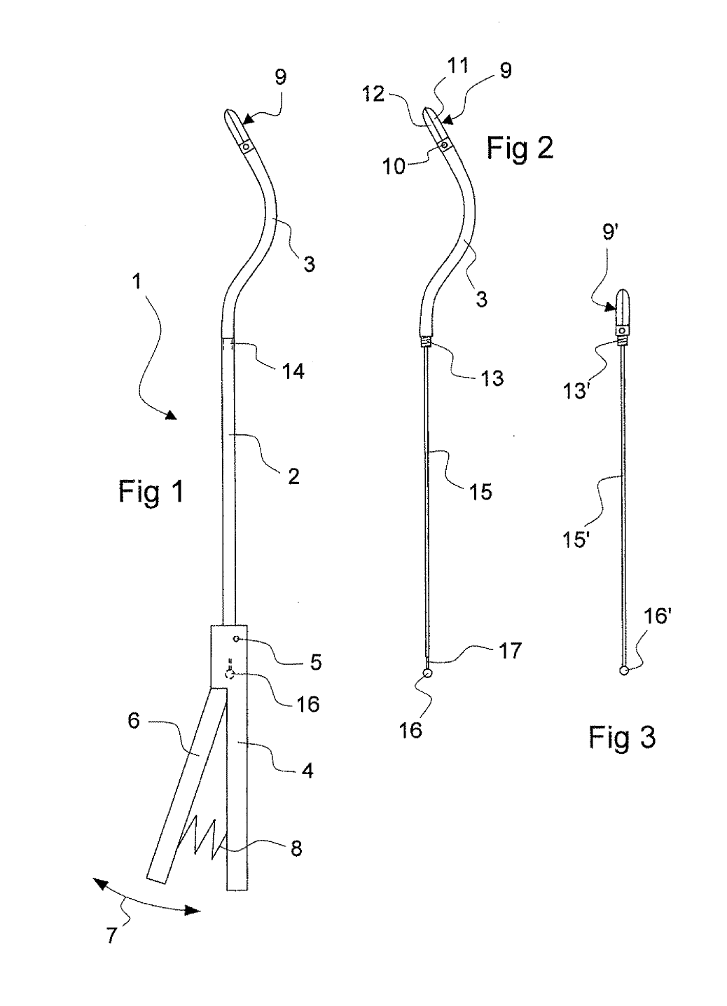

FIG. 1 shows a complete needle holder 1 according to an embodiment of the invention in side view. The needle holder 1 has a shaft embodied as a tube, with a straight proximal shaft section 2 and a curved distal shaft section 3.

A handle 4 is attached to the proximal end of the proximal shaft section 2, which handle is embodied as a fixed handle part and on which a moveable handle part 6 is supported which can be pivoted about an axis 5 according to the arrow 7, which handle part is supported with a spring 8 with respect to the fixed handle 4. This construction produces the typical form of an inline handle that is particularly suitable for needle holders. However, another type of handle can also be used, such as, for example, a pistol-form handle.

The distal shaft section 3 of the shaft 2, 3 is shown separately in FIG. 2. Clamping jaws 9 are discernible on the distal end of the distal shaft section 3, which clamping jaws are formed with two jaw parts 11, 12 mounted so as to be pivotable about an axis 10.

A connecting element in the form of a threaded part 13 with external thread is arranged on the proximal end of the distal shaft section 3, which connecting element is used for screwing in an internal thread 14 at the distal end of the proximal shaft section 2.

A rod 15 is attached to the clamping jaws 9 for the actuation thereof, runs through the tubular distal shaft section 3 and projects beyond this in the proximal direction, as is shown in FIG. 2. The rod 15 is provided on its proximal end with a cam in the form of a sphere 16, which can be screwed with a threaded part 17 into the proximal end of the rod 15.

As FIG. 1 shows, the assembly shown in FIG. 2 can be placed on the distal end of the proximal shaft section 2 of the shaft and firmly coupled by screwing in the threaded part 13 into the internal thread 14. Thereby the proximally projecting end of the rod 15 is thereby pushed through the proximal shaft section 2 of the shaft in the proximal direction, until the sphere 16 is in the position shown by a dashed line in FIG. 1, in which it is coupled in a manner not shown with the internal actuating mechanism of the handle 4, namely usually in such a manner that when the parts 4 and 6 of the handle 4 are compressed, the rod 15 is drawn in the proximal direction, which results in a closing movement of the jaw parts 11 and 12.

For reasons of a clear representation, in FIGS. 1 and 2 the clamping jaws 9 and the handle 4 are arranged on the shaft 2, 3 such that they open and close in the drawing plane. The corresponding axes 10 and 5 are thereby perpendicular to the drawing plane. For reasons of better handling, it can be advantageous to arrange the clamping jaws 9 and/or the handle 4 on the shaft at different angles, e.g., rotated by 90° compared to the position shown.

If the assembly essentially composed of the distal shaft section 3 and the rod 15, shown in FIG. 2, is removed, in its place another assembly in the form of clamping jaws 9′ shown in FIG. 3 with a rod 15′ arranged thereon with a sphere 16′ can be attached to the proximal shaft section 2 of the needle holder 1. The clamping jaws 9′ correspond to the clamping jaws 9 of the construction of FIG. 2. A threaded part 13′ is attached thereto, which corresponds to the threaded part 13 of FIG. 2. The assembly of FIG. 3 thus can be screwed on the proximal shaft section 2 instead of the assembly of FIG. 2, wherein the sphere 16′ instead of the sphere 16 is then coupled to the handle 4, 6. The clamp shown in FIG. 1, which is embodied with its curved distal shaft section 3 for work with several instruments in a common laparoscopic port, can thus be converted to a conventional needle holder construction, in which the clamping jaws 9′ rest directly on the distal end of the straight proximal shaft section 2.

The sphere 16′ is directly attached, e.g., welded, to the rod 15′ of the construction of FIG. 3. In series production, these components can all be produced with a constant length of the rod 15′ and thus with a constant spacing of the sphere 16′ from the clamping jaws 9′. These components then always fit the proximal shaft section 2 of the needle holder 1.

However, in the construction of FIG. 2, the rod 15 runs through the curved distal shaft section 3, in which considerable production tolerances can occur. The threaded part 17 is therefore advantageous, with which the spacing of the sphere 16 from the threaded part 13 can be adjusted to the measurement exactly fitting the proximal shaft section 2 of the needle holder 1, in order to be subsequently welded.

The needle holder 1, according to the configuration of FIGS. 1 and 2, is produced according to the method described below.

The straight proximal shaft section 2 of the shaft is prefabricated together with the handle 4, 5, 6, 8. Preferably, it can be taken from a production series that is produced together with the insert of FIG. 3 as a conventional straight needle holder.

The curved distal shaft region, which is shown in FIG. 2, is prefabricated separately. The clamping jaws 9 can likewise be taken from the series from which the clamping jaws 9′ of FIG. 3 also originate. Firstly, the rod 15 is attached to the clamping jaw 9. This is then placed through an initially still straight tube piece, at the distal end of which the clamping jaws 9 are attached, e.g., by welding. Then this tube piece, together with the rod 15 lying inside, is curved to form the curved distal shaft section 3. On the proximal end of the rod 15 a tapped hole is located, into which the threaded part 17 with the sphere 16 is screwed, until the sphere 16 has the desired spacing from the proximal end of the distal shaft section 3. Then, the threaded part 17 is welded in order to permanently fix the sphere 16 at its spacing.

Subsequently, the assembly thus formed and shown in FIG. 2 is installed in the needle holder 1 of FIG. 1, namely in the same manner as the assembly of FIG. 3, by screwing in the internal thread of the proximal shaft section 2 and by coupling the sphere 16 in the handle 4.

Instead of the attachment of the shaft sections 2 and 3 of the shaft to one another with the threaded parts 13 and 14, a different detachable coupling can be provided here, e.g., by means of bayonet or the like. However, it must be ensured thereby that the angular position of the curved distal shaft section 3 of the shaft to the handle 4 is always correctly aligned.

With the construction shown of FIGS. 1 and 2, the clamping jaws 9 are connected in a rotationally fixed manner, e.g., welded, to the distal shaft section 3 of the shaft. This results in a very simple and cost effective construction. To simplify the suturing operation, however, it would definitely be desirable to be able to rotate the clamping jaws 9 with respect to the distal shaft section 3. Then a corresponding pivot bearing would be necessary there and a rotary transmission from the handle 4 to the clamping jaws 9 could be carried out, e.g., by the torsionally stiff rod 15. Then a rotating actuator, which acts on the rod 15 accordingly, would have to be provided on the handle 4.

What is claimed is:

1. A laparoscopic needle holder comprising:
a tubular shaft, the shaft having a straight proximal shaft section and a curved distal shaft section;
clamping jaws disposed at a distal end of the shaft;
a handle disposed at a proximal end of the shaft; and
a rod that passes through a center of the shaft, the rod capable of being longitudinally displaced by the handle to actuate the clamping jaws,
wherein the straight proximal shaft section and the curved distal shaft section are detachably attached to one another.

2. The laparoscopic needle holder according to claim 1, wherein
the straight proximal shaft section and the curved distal shaft section are connected via a coupling, the coupling is disposed at a distal end of the proximal shaft section and is capable of accommodating the clamping jaws.

3. The laparoscopic needle holder according to claim 1, wherein
the clamping jaws are attached to the curved distal shaft section in a non-detachable manner.

4. The laparoscopic needle holder according to claim 1, wherein
the rod includes a thickened cam on a proximal end of the rod for coupling with the handle, the cam being capable of attaching to the rod such that a space between the proximal end of the rod and the cam is adjustable.

5. The laparoscopic needle holder according to claim 4, wherein
the cam is screwed onto the rod and fixed to the rod by welding.

6. A method for producing a laparoscopic needle holder having a shaft, clamping jaws, a rod and a handle, the shaft including a proximal shaft section and a distal shaft section that are detachably attached, and the rod including a cam for coupling with the handle, the method comprising:
inserting a prefabricated mounting unit including the clamping jaws and the rod into the distal shaft section;
attaching the clamping jaws to a distal end of the distal shaft section;
bending the distal shaft section with the rod inserted into the distal shaft section; and
attaching the proximal shaft section to the distal shaft section.

7. The method according to claim 6, wherein
the cam is adjusted to a desired spacing by screwing the cam onto the rod and is then attached to the rod by welding.

8. A laparoscopic needle holder comprising:
a tubular distal shaft section, the distal shaft section including a connecting element disposed on a proximal end for connecting to a distal end of a proximal shaft section and having a curved-shaped;
clamping jaws disposed at a distal end of the distal shaft section; and
a rod that passes through a center of the distal shaft section, the rod including a thickened cam disposed on an end of the rod and projecting proximally beyond the connecting element.
